(19) United States
(12) Patent Application Publication
(10) Pub. No.: US 2021/0177628 A1
(43) Pub. Date: Jun. 17, 2021

(54) Prosthetic Foot Cover System Using Pressure Chambers

(71) Applicant: Filiauer Composites LLC, Salt Lake City, UT (US)

(72) Inventors: Eric W. Rubie, Salt Lake City, UT (US); Jonathan A. Winegar, Bountiful, UT (US)

(21) Appl. No.: 17/124,470

(22) Filed: Dec. 16, 2020

Related U.S. Application Data
(60) Provisional application No. 62/948,621, filed on Dec. 16, 2019.
(60) Provisional application No. 62/948,611, filed on Dec. 16, 2019.

(51) Int. Cl. A61F 2/66; A61F 2/80; A61B 5/11; A61B 5/00

(52) U.S. Cl. CPC A61F 2/66; A61F 2/80; A61B 5/1118; A61F 2002/805; A61B 5/6811; A61F 2002/6642; A61F 2002/665; A61B 5/112

(57) ABSTRACT

A prosthetic foot cover device with a pressure chamber is described. The prosthetic foot cover may include a lattice framework formed to replace a human foot. An open cavity may be formed in the lattice framework, which is sized and shaped to contain a prosthetic foot. A pressure chamber may be disposed in the lattice framework. A first one-way check valve is provided for the pressure chamber to allow air to be expelled from the pressure chamber and to generate a negative pressure within the pressure chamber as an amputee uses the prosthetic foot and the lattice framework. A remnant limb socket may be connected to the pressure chamber. A negative pressure may be developed in the remnant limb socket as air is pulled from the remnant limb socket into the pressure chamber. A method for additive manufacture of a prosthetic foot cover device is also described.

### PRIORITY DATA

This application claims benefit of U.S. Provisional Patent Application Ser. No. 62/948,611, filed Dec. 16, 2019 and U.S. Provisional Patent Application Ser. No. 62/948,621, filed Dec. 16, 2019, both of which are incorporated herein by reference.

### BACKGROUND

Prostheses (or prosthetics) are artificial devices that replace body parts (e.g., fingers, hands, arms, legs). Generally, prostheses may be used to replace body parts lost by injury or missing from birth. The quality of prostheses has greatly improved in recent years. For example, a prosthetic limb may be molded to have the same shape and density as the person's remaining limb. In addition, elastomeric polymer skins may be used to form the prosthetic limb and give the prosthetic limb a life-like appearance. As another example, improvements in prosthetic limbs may allow for increased feedback and movement.

However, prosthetic limbs still present numerous challenges, particularly in the area of looking and performing as the actual human limbs being replaced. An intact human foot, connected to its ankle, travels through stance and swing phases of a gait cycle during each stride of motion, whether the motion involves walking, jogging, or running. In order to provide higher performance prosthetics, prosthetic feet made of composite materials are often used to provide the energy return characteristics desired by an amputee. For example, leaf spring composite prosthetic feet may provide desirable characteristics for a foot amputee.

In order to improve the appearance of prosthetic feet, the prosthetic feet are often covered with a cosmetic foot shell that forms an open cavity around the functional prosthetic foot structure. Existing foot shells may be thin-wall hollow shells that do not enhance the prosthetic foot function and often diminish prosthetic foot response. For example, these existing foot shells may be made of polyurethane, PVC (polyvinyl chloride), silicone or other plastics and rubber substitutes. One common problem with foot shells is the internal cavity of the foot shell does not securely hold the prosthetic foot, composite leaf spring foot or other prosthetic foot elements in place which causes the foot to be loosely connected to the foot shell and amputee's shoe. This lack of a secure connection may impede feedback to the user and decrease overall performance of the prosthetic foot.

### BRIEF DESCRIPTION OF THE DRAWINGS

FIG. 1 illustrates an example of a prosthetic foot cover device for a prosthetic foot.

FIG. 2A illustrates an example of one half of a lattice framework that is separate from an outer membrane layer.

FIG. 2B illustrates an example of a side view of a full prosthetic foot cover.

FIG. 2C illustrates an example of an isometric view of a full prosthetic foot cover.

FIG. 3 illustrates a prosthetic foot to show the relation between the prosthetic foot, the lattice framework and the outer membrane layer.

FIG. 4 illustrates an example of an outer membrane layer is joined with the lattice framework.

FIG. 5 illustrates an example of lattice sub-shapes and sub-areas which may be formed into a lattice structure of non-uniform density.

FIG. 6 illustrates an example of an outer membrane layer and lattice core formed in single piece or solid shell.

FIG. 7 illustrates an example a prosthetic foot cover device where the lattice framework conforms to the outer membrane layer with a defined thickness.

FIG. 8 illustrates a configuration similar to FIG. 7 where the lattice framework is relatively sparse or less dense as compared to the density of the lattice framework in FIG. 7.

FIG. 9 illustrates the lattice framework where the lattice sub-structures are a plurality of curved plates attached to the outer membrane layer.

FIG. 10 illustrates a side view of a foot framework where the sub-structures are an open cavity design with an internal support structure.

FIG. 11 illustrates a configuration of a prosthetic foot cover device for a prosthetic foot that includes pressure chambers and one-way valves.

FIG. 12A illustrates that the lattice structure may contain one or more chambers with air flow valves in the lattice framework or near to the lattice framework, and the chambers may be under the heel, toe, or arch.

FIG. 12B illustrates a heel bumper with air valves to enable adjustment of the pressure in the heel bumper pressure chamber.

FIG. 13A illustrates that the lattice structure may contain one or more sealed chambers in the lattice framework or near to the lattice framework.

FIG. 13B illustrates a heel bumper integrated into the foot cover in the form of a sealed chamber and lattice.

FIG. 14 is a flowchart illustrating a method for custom creation of a lattice framework.

FIG. 15 illustrates a system to generate a custom architecture of a lattice framework via additive manufacturing to create a custom prosthesis.

FIG. 16A illustrates an example of a prosthetic foot and leg.

FIG. 16B is a side view of a prosthetic foot cover, prosthetic ankle and prosthetic leg using a lattice framework in one example.

FIG. 16C is a perspective view illustrating an example of a prosthetic foot cover, prosthetic ankle and prosthetic leg using a lattice framework.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure are to be considered within the scope of the description.

The present technology provides a prosthetic foot cover with an integrated framework, lattice framework(s) or lattice networks. The integrated frameworks and lattices can be made using an additive manufacturing process or a 3D (three-dimensional) printing process. The integrated lattice frameworks may be made of an elastic polymer, another flexible material, or a semi-rigid polymer material that can used in a 3D printing process.

Additive manufacturing (AM) may be used to create intricate lattice structures to meet a wide variety of design requirements in a prosthetic limb. AM offers the opportunity to create generative lattice structures for a variety of prosthetic foot designs. Intricate lattice features that were once difficult to produce using injection molding can now be integrated into prosthetic limbs to optimize performance. The benefits of lattice structures can be further exploited to enable mass customization of prosthetic limbs and more specifically prosthetic feet.

Most prosthetic feet have been covered with a cosmetic foot shell that creates an open cavity around the foot structure. In the past, foot shells have been thin-wall hollow shells that do not enhance the prosthetic foot function and often diminish prosthetic foot response. A common problem with foot shells is that the internal cavity does not securely hold the prosthetic foot elements (e.g., composite leaf spring) of a prosthetic foot in place which may cause the foot to only be loosely connected to the cosmetic foot shell and may impede feedback to the user. Therefore, a secondary foam filler may be used to fix the prosthetic foot in place within the foot shell in order to remedy this limitation, but this foam filler tends to inhibit the movement of the prosthetic foot inside the shell.

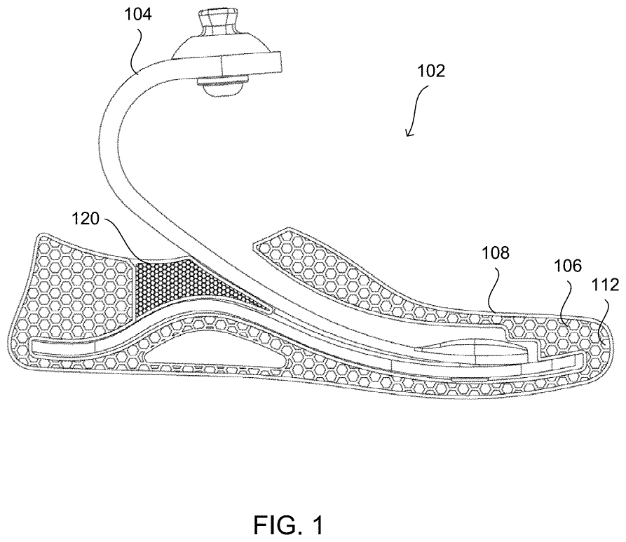

FIG. 1 illustrates a prosthetic foot cover 102 for a prosthetic foot 104. For example, the prosthetic foot may be a composite leaf spring foot. A lattice framework 106 may be provided and the lattice framework for a prosthetic foot may be formed in the three-dimensional shape or cross-section of a human foot or another shape approximating a human foot. The lattice chambers 112 or lattice sub-elements may be formed in any number of shapes. The hexagonal shape shown in FIG. 1 may be one shape for a lattice framework but any other geometric shapes (e.g., rectangles, triangles, circles, diamonds, hexagons, octagons, irregular polygons, other geometric shapes, 3D spine shapes, complex repetitive shapes, organic shapes (e.g., irregular, asymmetric, or curvy), fractal shapes, etc.) may be used. In this configuration, a thin-wall lattice membrane may also be used to replace the solid foam cores of prior foot shells. A membrane design with a lattice core makes producing a lighter and more responsive foot shell feasible. Thus, an amputee may have a prosthetic foot that has the volume of a human foot but the prosthetic foot may still be light weight.

An outer membrane layer 108 may be disposed over the lattice framework 106 to cover and support the lattice framework 106. The prosthetic foot cover may have a thin-wall lattice membrane to cover the lattice framework for a prosthetic foot. The outer membrane layer 108 may also be removable. In another configuration, the outer membrane layer 108 may be fixed to or additively formed with the lattice framework 106. The lattice structure of the lattice framework 106 may be a uniform lattice where the lattice sub-shapes are uniform in size. Alternatively, the lattice framework 106 may include lattices of varying sizes 120, as will be discussed in more detail later.

In the past, passive prosthetic foot systems have had limited potential to adjust the functional characteristics of the prosthetic system since the systems have often relied on elastomeric inserts in a foot cover to achieve a desired response. In the present technology, the foot cover can be used to adjust the prosthetic foot and may be utilized as a functional member of the foot system to provide enhanced performance to an amputee. The foot cover can also offer a means to regulate and control the foot system by controlling the density of the lattice that is used. The design of lattice foot structure may be tailored to provide a specific compliance (or stiffness) so that the entire gait cycle is optimized to the user's needs.

FIG. 1 also illustrates that heel dampeners and/or bumpers 120 can utilize a lattice structure design that is integrated into the foot cover. The use of a heel dampener or bumper 120 as part of the prosthetic foot cover 102 may be used to change the operating characteristics of the overall prosthetic foot. For example, the heel dampener may change the operating characteristics of a composite leaf spring foot.

In one configuration of the technology, the foot cover may be additively manufactured by selective laser sintering (SLS) which fuses a polymer powder or plastic powder (e.g., nylon or polyamide) laid down in powder layers into a foot cover structure using a computer guided laser that activates layers of the powder. The present technology allows the lattice foot framework to be created using SLS but since the outer membrane layer is separate, this allows the outer membrane layer to be removed and the polymer powder can be easily emptied from the lattice. Otherwise, if the outer membrane layer and lattice are manufactured together using SLS, the powder and its added weight are quite difficult to remove from the foot cover. The outer membrane layer can be removed after the outer membrane is manufactured in the same powder volume or powder stack, or the outer membrane layer can be manufactured separately so that the powder can be removed from lattice framework.

Alternatively, the outer membrane layer can be exchanged for any outer membrane layer that is made by any manufacturing process (e.g., additive, injection, or otherwise). This may provide any cosmetic appearance that an amputee desires. For example, a foot cover can be provided that is larger than the lattice framework. Then the foot cover can be placed over the lattice framework and the foot cover can be heated (e.g., boiled in water or using heated air) to shrink the foot cover to fit over the lattice framework.

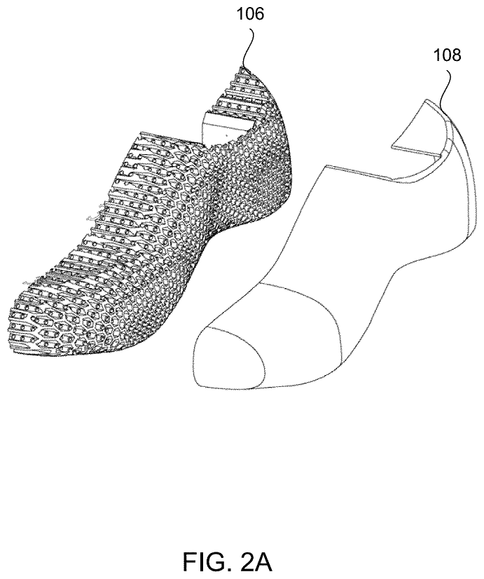

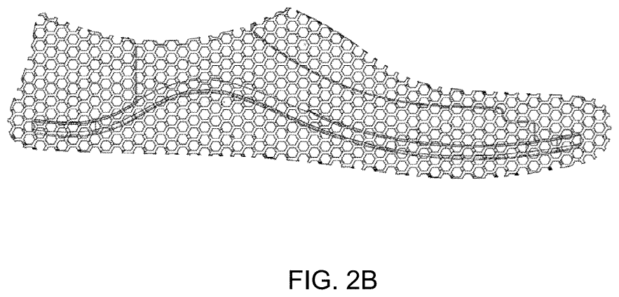

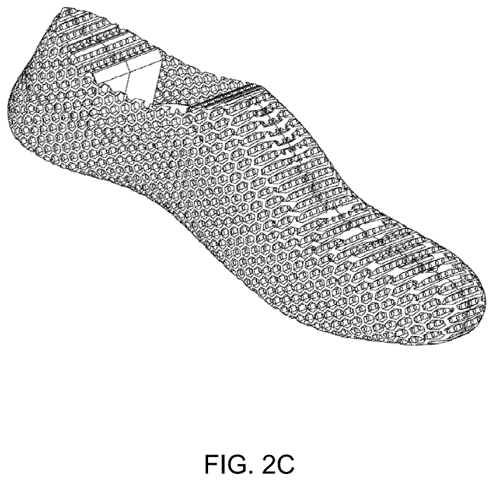

FIG. 2A illustrates that a lattice framework 106 may be removed and/or separated from the outer membrane layer 108. This may allow for the lattice frameworks 106 of differing densities to be inserted into the outer membrane layer. If an amputee needs a greater density in the lattice framework 106 for performing a specific activity, such as running, a lattice framework 106 with a greater density (e.g., smaller sub-lattice structure size) may be inserted. If an amputee wants a lattice framework 106 for walking, then a lattice framework 106 may be inserted that has less overall density. Similarly, the density of the lattice framework may be modified depending on the size or weight of the amputee. An amputee that is of greater mass may need a lattice framework that is denser and the amputee may be willing to carry the extra weight for the extra support. An amputee that has less mass, such as a youth, may want less support from a lattice framework and also want the lower weight that comes with the lower density lattice framework.

FIG. 2B illustrates an example of a side view of a full prosthetic foot cover without a membrane cover. FIG. 2C illustrates an example of an isometric view of a full prosthetic foot cover (i.e., two halves joined together).

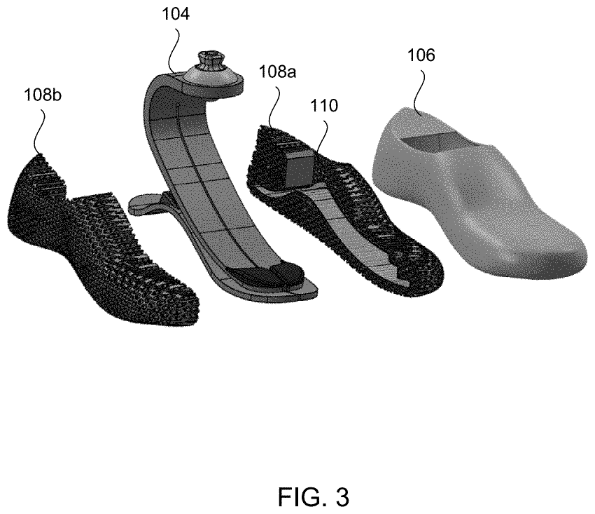

FIG. 3 further illustrates that a prosthetic foot 104 may be inserted into a lattice framework that has two halves 108a, 108b. An inner cavity 110 may be formed in the lattice structure which is sized and shaped to contain or receive the prosthetic foot 104. For example, the inner cavity 110 may have an opening that is sized to receive a composite leaf spring foot. More specifically, the prosthetic foot 104 may be pressed into the inner cavity 110 in a lateral or sideways direction. The lattice framework 106 may be provided in two halves so that the prosthetic foot can be slipped into the lattice framework 106 easily. Alternatively, the lattice framework may be a single piece covering the composite leaf spring foot 104.

The lattice framework 108a, 108b and the prosthetic foot 104 may be inserted into or covered by the outer membrane layer 106. The lattice framework 108a, 108b and the outer membrane layer 106 may be a single unit, as illustrated, in order to cover the prosthetic foot 104. For example, the lattice framework 108a, 108b and the outer membrane layer 106 may work with the prosthetic foot 104 as a system to tune the total response of the prosthetic foot.

FIG. 3 illustrates that the outer membrane layer 108 may have a first half or left half of the outer membrane layer 108. In addition, a right half (not shown) may be provided to cover a right lattice framework. In an alternative configuration, the lattice foot framework 106 may be provided in two halves but the outer membrane layer may be a single piece that covers both halves of the lattice framework.

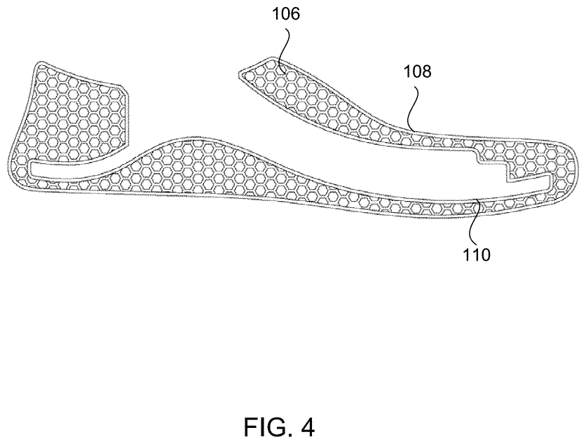

FIG. 4 illustrates a side orthogonal view of the lattice framework. The outer membrane 108 may be formed as part of the lattice framework. In addition, the outer membrane 108 may also wrap around to form an inner membrane layer 110 which provides additional support for the prosthetic foot.

The foot cover may be composed of lattice structures with mechanical properties that can be used to enhance the functional characteristics and responsiveness of the prosthetic foot. For example, the foot cover may have a lattice structure that become an integral part of the prosthetic foot. There are a wide variety of design possibilities when utilizing lattice structures, and the lattice structures may enable a specific desired response to be integrated into the foot cover. One configuration of this technology incorporates a uniform lattice structure which provides a predictable response due to the homogeneous density. Another variation uses a combination of different density lattices of various densities to generate a variable dynamic response for optimizing the functional characteristics for the user.

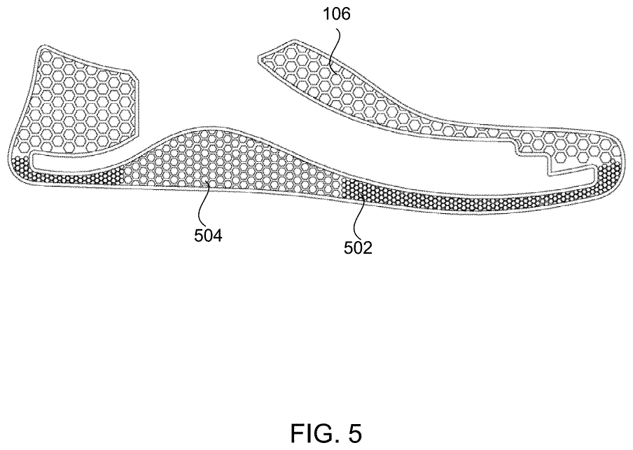

FIG. 5 illustrates that the lattice sub-shapes may be formed into separate areas in a lattice structure so that the overall lattice structure has a non-uniform density or varying density. For example, the lattice structure may have variable densities to generate a variable dynamic response and to optimize functional characteristics for a user. In one configuration, the sub-shapes may be smaller in areas 502 of the lattice structure where more support or strength is needed or larger in separate areas 504 where less support is needed.

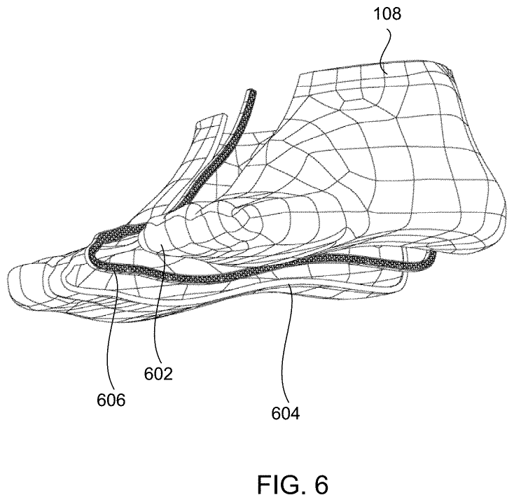

FIG. 6 illustrates a configuration of an outer membrane layer 108 that is designed to appear more anatomically accurate. The outer membrane layer 108 or solid shell may be formed from a single membrane piece and may provide a more anatomically accurate arch 604, set of toes 602, other anatomically accurate aspects of a human foot. FIG. 6 illustrates a cross-sectional portion 606 of the outer membrane layer 108 (for explanatory purposes) that illustrates a lattice network that is attached to the single piece of the more anatomically accurate outer membrane layer 108 or more anatomically accurate foot cover. In one configuration an inner membrane layer may also be included.

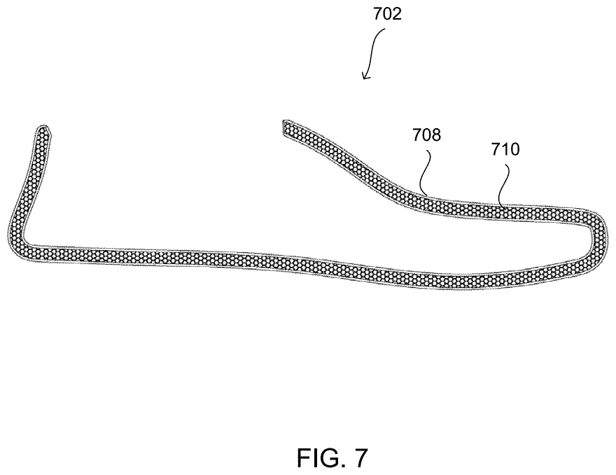

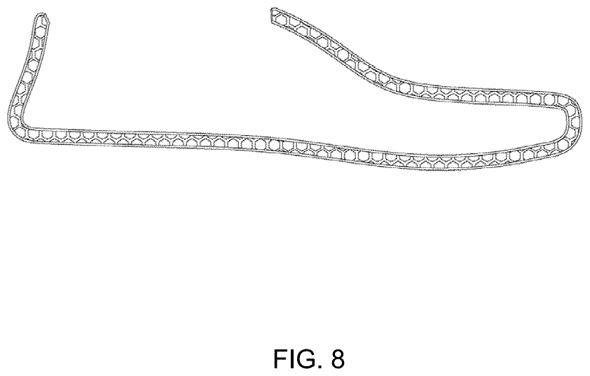

FIG. 7 illustrates a configuration of a prosthetic foot cover device 702 where the lattice framework 710 conforms to the perimeter foot shape that is represented by the outer membrane layer 708. This means the lattice framework 710 is shaped to enclose a volume of a human foot or volume of an anatomical foot shape. In this case, the lattice framework may provide functional support, resilience or cushioning while being just a few millimeters to several centimeters thick, while substantially conforming to the shape of the outer membrane layer 708. FIG. 7 also illustrates that the lattice framework is a relatively dense lattice framework (as compared to earlier figures). FIG. 8 illustrates a configuration similar to FIG. 7 where the lattice framework is relatively sparse or less dense as compared to the density of the lattice framework in FIG. 7.

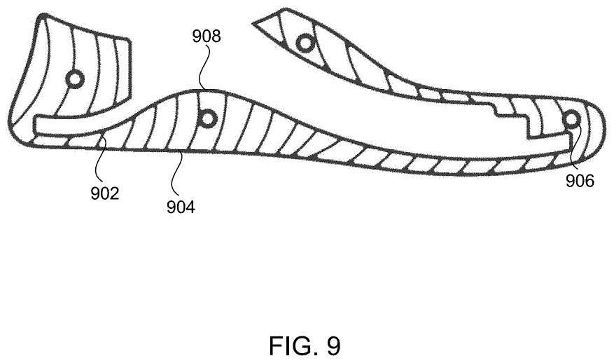

FIG. 9 illustrates the lattice framework where the lattice sub-structures are a plurality of arced plates 902 or curved sheets attached to the outer support layer 904 and an inner support layer 908. Alternatively, this lattice framework may be supported by an outer support layer 904 that is a mesh and the lattice framework may be covered by the outer support layer. For example, the outer support layer 902 may be a mesh of triangular, rectangular, circular, diamond, hexagon, octagon, circle, half moon, irregular polygons, 3D spine shapes, complex repetitive shapes, organic shapes, fractal shapes or other geometric shapes. Connector holes 906 are also illustrated that may be used to connect two halves of the lattice framework in FIG. 1 using threaded or press-fit connectors.

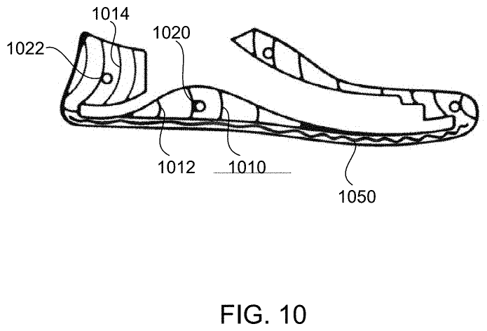

FIG. 10 illustrates a side view of open cavity design with an internal support structure. The open cavity design may include a lattice framework where the lattice sub-structures are a plurality of curved support structures 1010, 1012, 1014, curved plates, or curved slabs attached to the outer membrane layer. In addition, the curved supports may be attached to supporting cylinders 1020, 1022 or rods. Other supporting structures such as a curved sheet in a sine wave 1050 may be included to support the ball of the foot.

The open cavity design may be lighter than the lattice framework depending on the internal support used. The open cavity design may accommodate a variety of internal structures. The internal structures may be a variety of configurations, such as leaf springs, which can be altered to meet design parameters for a specific amputee. In the case of using resilient leaf springs for the support structure, the resilient leaf springs may offer better cyclic performance (i.e., long-term durability) over time.

The use of a lattice framework with the foot cover may enable the integration of additional design features and/or devices that enable the regulation and control of the prosthesis. One example of a device that can be integrated into the foot cover is a vacuum pump which creates a negative pressure or pulls vacuum on the residual limb to maintain the amputee's intimate contact with a socket for supporting the amputee's remnant limb. A vacuum pump device can be integrated into the foot cover so that during the stance phase when the foot is loaded, a chamber is compressed to expel air, and then when unloading occurs, vacuum or negative pressure is drawn on the limb when the chamber expands.

Using the vacuum device with the foot cover may be beneficial for several reasons. Integration of a vacuum pump into the foot cover may allow the spring element of a prosthetic foot to activate the pump when the amputee walks, runs or moves on the prosthetic foot. In one arrangement, the deformation of the spring element compresses the vacuum chamber or pressure chamber which expels air through a one-way check valve. Once the chamber is fully compressed, pressure is then released from the spring element of the prosthetic foot. Next, the deformed vacuum chamber may pull vacuum when the vacuum chamber or pressure chamber expands back toward the less compressed state thru another one-way check valve connected to the socket via appropriately routed hosing.

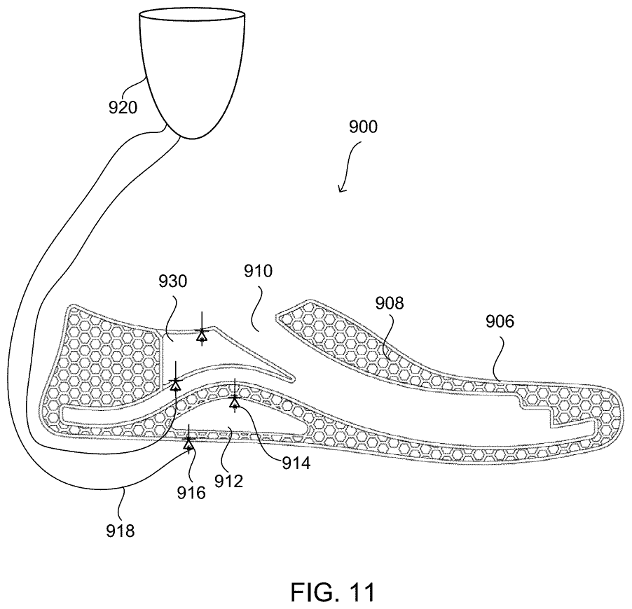

FIG. 11 illustrates another configuration of a prosthetic foot cover device 900 for a prosthetic foot. The prosthetic foot cover device 900 may include a lattice framework 908 formed in the shape of a human foot. An open cavity 910 may be formed in the lattice structure which is sized and shaped to contain the prosthetic foot, and the open cavity 910 may have an opening to receive, for example, a composite leaf spring foot (illustrated earlier).

A pressure chamber 912 may also be disposed in the lattice framework 908. The pressure chamber 912 may be used to generate a negative pressure as an amputee walks using the prosthetic foot and the lattice framework. A first one-way check valve 914 may be associated with the pressure chamber 912 to allow air to be expelled from the pressure chamber to the outside or outside atmosphere, when an amputee walks, runs or moves on the prosthetic foot. A second one-way check valve 916 may be associated with the pressure chamber 912 to allow air to be drawn into the pressure chamber.

A remnant limb socket 920 may be connected to the second one-way valve 916 using a conduit 918 or tubing, and a negative pressure or vacuum maybe developed in the pressure chamber 912 and remnant limb socket 920 as air is pulled from the remnant limb socket 920 through the pressure chamber 912. The tubing 918 may be plastic tubing, metal tubing or series of chambers to route air between the remnant limb socket 920 and the second one-way check valve 916. An outer membrane layer 906 may be disposed over the lattice framework 908 to cover and support the lattice framework, and the outer membrane layer 906 may be removable. A second pressure chamber 930 may also operate to provide additional or alternative negative pressure using a similar configuration as the configuration described in FIG. 11.

Additional configurations of the technology may include assistive mechanical devices (e.g., mechanical pumps) to facilitate higher levels of vacuum. For example, the prosthetic foot device may further include an assistive negative pressure pump that is mechanically powered by movement of a vertical compression device in the leg or a mechanical knee or a battery powered assistive negative pressure pump to withdraw air from the pressure chamber 912 as an amputee walks on the foot. The use of the pump can create a negative pressure or vacuum in the chamber which can be used to create negative pressure in the remnant limb socket 920, as described earlier. The negative pressure in the socket for the remnant limb assists with keeping the socket in place while the prosthetic foot and/or leg system is used.

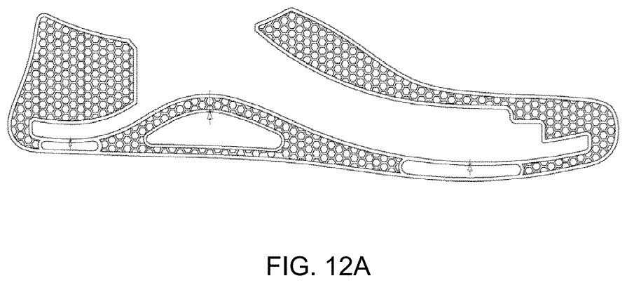

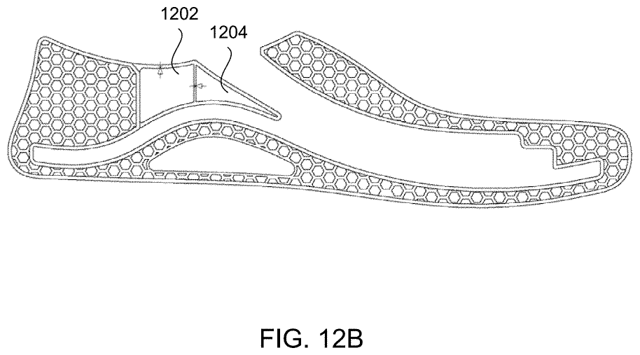

FIG. 12A illustrates another configuration of the technology which has one or more pressure chambers and one or more one-way valves placed strategically under the heel, toe or arch, and the hollow chambers may be designed to compress and expand to generate vacuum or negative pressure. Each chamber may have one or two valves as described in FIG. 11 and operate to generate a negative pressure in a receptacle or socket for a remnant limb of an amputee. Further, any number of additional chambers can be used in combination to reach higher vacuum levels.

FIG. 12B illustrates a heel bumper with air valves to enable adjustment of the pressure in the heel bumper chamber(s) 1202, 1204. The air valves may be mounted in the walls of the heel bumper chambers 1202, 1204. The amputee may adjust the heel response by increasing or decreasing the pressure (e.g., air pressure) in a first pressure chamber 1202. The pressure in a second chamber 1204 may be set to a different pressure than a first chamber to change the response of a bumper near the prosthetic foot and thus change the response of the heel of the prosthetic foot. Increasing the chamber pressure may create a faster response which would be suitable for more active users and conversely, decreasing the chamber pressure may attenuate shocks better and slow down the heel reaction which would be suitable for a less active user. As a result, the pressure chamber may replace solid rubber bumpers where two or three levels of dampening (e.g., high, medium, low bumpers) have been provided to amputees. Instead, an amputee may select the amount of dampening desired by changing the pressure in the pressure chamber(s) replacing the heel bumper.

Benefit may be gained by the ability to conceal the pumping device or negative pressure device inside the foot cover, so that no external device is needed. External devices may be undesirable since they can protrude and stick out from the prosthetic system in unnatural ways, as is the case with most prosthetic foot products with pumping systems that existed prior to the present technology. The vacuum device and pressure chamber(s) of this technology can be contained in a cavity and/or regions of the foot cover and concealed from view to create a more natural looking prosthesis.

The foot cover may also house structures, features and/or devices that may regulate and control shock absorption. In the past, prosthetic feet have utilized external apparatuses for shock absorption but these external devices have added significant weight and complexity to the foot systems. The present technology may use sealed chambers (e.g., sealed hollow chambers at atmospheric pressure or sealed pressurized chambers) placed strategically under a heel and toe which are designed to compress and absorb shock. These shock absorbing chambers can have valves and pressure regulators which can be adjusted and tuned to meet the user needs.

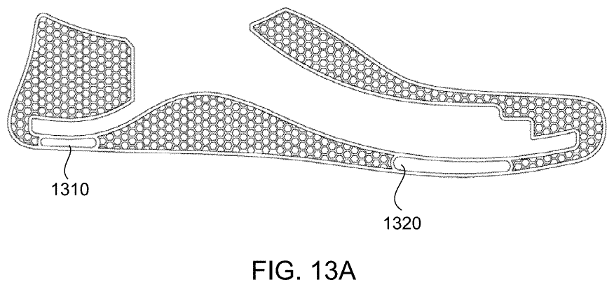

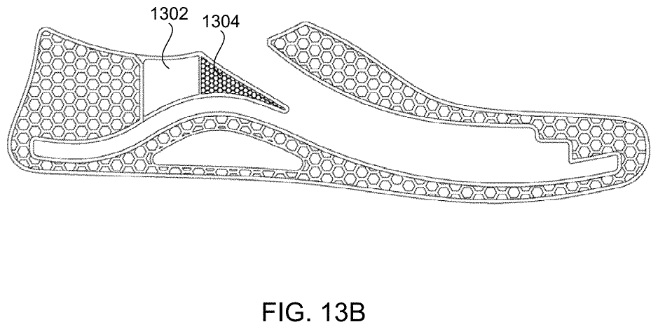

FIG. 13A illustrates that the lattice structure may contain one or more sealed chambers 1310, 1320 in the lattice framework or near to the lattice framework to provide cushioning for the prosthetic foot. The sealed chamber(s) 1310, 1320 (e.g., pressure chambers) may be in proximity to or under a heel, a ball or a toe of the prosthetic foot to absorb shock, provide cushioning and/or otherwise support the prosthetic foot. The sealed chambers may be sealed at atmospheric pressure.

FIG. 13B illustrates a heel bumper integrated into the foot cover in the form of a sealed chamber 1302 adjacent to a lattice bumper 1304. Thus, the foot cover may integrate with one or more heel dampeners to control the heel response. The areas used for the sealed chamber 1302 and the lattice bumper 1304 could also be switched

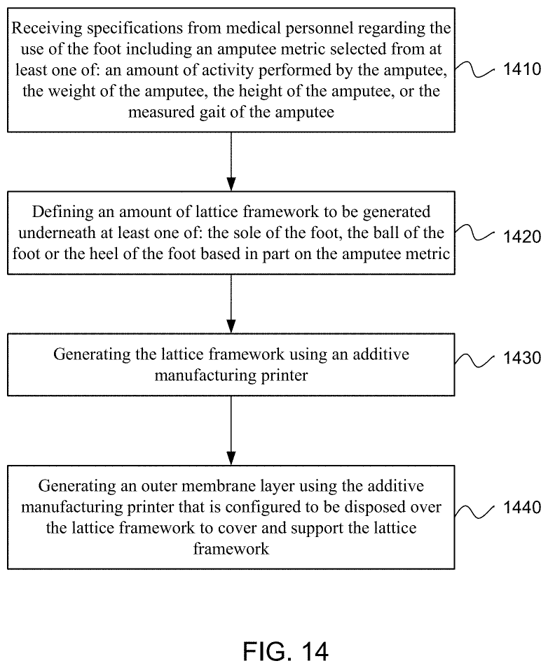

FIG. 14 is a flow chart illustrating a method for custom creation of a lattice framework. The method may include operations for additive manufacture of a prosthetic foot cover device. An initial operation may be receiving specifications at a measurement computing device and from medical personnel regarding the use of the foot, as in block 1410. The specification may include defining an amputee metric. One amputee metric may be physical user data obtained from a digital scanner or digital sensor (e.g., foot size, foot shape, leg length, external structure, body shape, body size, skeletal structure etc.). The digital scanner may be a camera, on optical scanner, pressure sensors, Mill, or any other known scanner to obtain the physical user data.

An amount of activity performed by the amputee may also be obtained. For example, the amount of activity may be high, medium, low. Alternatively, the amount of activity may be defined using medical definitions for amputees. In addition, the weight of the amputee, the height of the amputee, or the measured gait of the amputee may be obtained. The gait of the amputee may include defining a length of a gait, where an amputee's gait and stance causes them to place weight on a prosthetic foot, or other biomechanical aspects of an amputee's gait.

Another operation in the method may be analyzing the input data to define an architecture of lattice framework to be generated and manufactured for use by the amputee, as in block 1420. The analysis may generate an open lattice framework or lattice cover matches the amputee's gait. For example, the open lattice framework may be stronger in areas where the amputee needs more support or corrective structures may be built into the open lattice framework to correct for any missing anatomical structures in an amputee's leg. In another example, the amount of activity of an amputee may be translated to a stiffness, resilience or density of the lattice framework.

The lattice framework to be generated may be a lattice framework to be used underneath the prosthetic foot or at the underside of the prosthetic device of the amputee. For example, the lattice framework may be generated for a sole of the foot, a ball of the foot or a heel of the foot based in part on the amputee metric or input data regarding the user of the prosthetic foot. A further operation may be generating the lattice framework using an additive manufacturing printer, as in block 1430.

The lattice framework may be integrated into the prosthesis to form a customized system specific to the amputee. For example, the lattice framework may be combined into a foot cover, a shoe, or other specialized foot construction. Optionally, an outer membrane layer may be generated using the additive manufacturing printer that is configured to be disposed over the lattice framework to cover and/or support the lattice framework, as in block 1440. This foot cover can provide variable tunable lattice portions, variable stiffness, and customizing of the lattice to fit amputee and/or the model of prosthetic foot being used.

In addition to creating a lattice framework, a lattice framework can also be incorporated in activity specific footwear and soles created using a lattice structure, which may enable better adaption by amputees. Amputees may no longer be restricted to only wearing existing types of shoes over a foot cover. For example, instead of providing a foot cover that looks like a human foot, a foot cover that looks like a shoe and has a sole with a lattice structure may be manufactured. In another example, the foot cover may have the appearance of a foot and a shoe may be worn over the foot cover but both the lattice framework in the foot cover and the shoe sole may be tuned for the individual amputee and the activities in which the amputee participates. Thus, customized sport soles can be designed for activity specific products to optimize user performance.

Prosthetic users can benefit from customization where the clinician may specify the desired compliance of the system for the user based on the results of a clinical evaluation. Therefore, the clinician can digitally select the design features to be incorporated into the prosthetic foot cover to achieve the desired foot response and then use generative design software to optimize the prosthetic foot cover for the user. The clinician can further adjust and fine tune the prosthetic device by creating a customized foot cover for the patient.

The lattice frameworks can also be used for creating mid-soles and outer soles which are designed for specific uses and for the shoes which are used with the prosthetic foot systems. With the advent of mass customization in shoes, the midsole of shoes may be generated and integrate into the overall prosthetic foot system. For example, sport specific soles may incorporate a lattice structure tuned for participating in sporting events and such soles can be fabricated via additive manufacturing (AM).

The ability to create custom foot lattice structure enables the selective integration of complex lattice structures into a foot cover to regulate and control foot performance. For example, the lattice structure can be discretely positioned throughout the prosthetic foot cover to achieve a desired response according to the user's needs and to produce a controlled response.

An Infinitely Variable Lattice Structure (IVLS) also provides the ability to selectively modify and alter the density/geometry of regions of the lattice framework to achieve the desired functional characteristics of the prosthetic foot.

The rollover characteristics of the foot system can be regulated and tuned by strategically positioning the lattice structure throughout the foot cover. For example, the tuning of the prosthetic foot may be considered a Lattice Tuned Design (LTD). Hollow chambers or highly compliant lattice structures can be used on the plantar surface of the cover to function as shock absorbers.

Another aspect of passive prosthetic foot systems is that there is limited potential to adjust the functional characteristics of prior prosthetic foot covers since they often rely on elastomeric inserts to achieve a desired response. Accordingly, the present foot cover can be used to adjust system characteristics in light the prosthetic foot used, and the foot cover can be used as a functional member of the foot system which provides enhanced performance.

The present technology consolidates many individual parts in a single assembly and this in turn may reduce the part count of the prosthetic foot and result in better reliability. For example, a prosthetic foot cover, a lattice, a negative pressure pump and a customizable heel dampener may be contained within one prosthetic foot cover or within a monolithic device created using a single additive manufacturing or printing process.

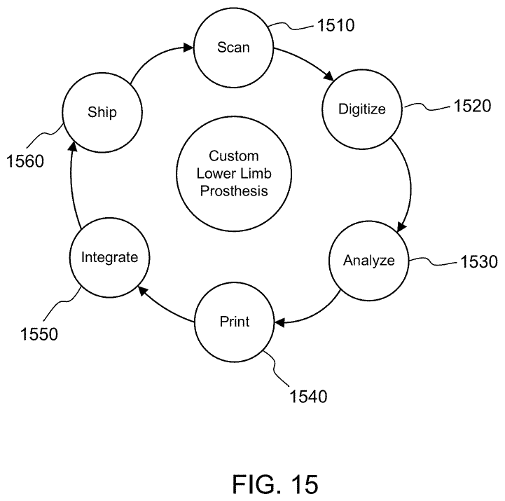

FIG. 15 illustrates a system to generate a custom architecture of a lattice framework via additive manufacturing to create a custom prosthesis. Initially, information about a prosthesis user may be scanned by a scanning device 1510 using electronic sensors. Amputee metrics may be physical user data obtained from a digital scanner as discussed earlier. For example, gait, foot size, foot shape, leg length, external body structure, body shape, body size, skeletal structure, etc. may be scanned. The digital scanner may be a camera, an optical scanner, pressure sensors on a foot plate, an Mill, or any other known networkable electronic scanner to obtain the physical user data. The scanned information may be digitized 1520 by a computing device attached to a sensor and stored in an electronic file locally or in a centralized cloud storage location. Other survey information such as an amount of activity performed by the amputee, an amputee weight, an amputee height or an amputee gait may also be digitized.

An analysis module 1530 on a computing device (e.g., a laptop or workstation) may analyze the digitized data about the amputee and determine a lattice framework to be made as a foot cover to a prosthetic foot. For example, a larger person may have a lattice framework with thicker walls for the cells of the lattice. In another example, an amputee who plays sports (e.g., tennis) may have the ball of the foot reinforced with a denser lattice, etc. The lattice framework may then be printed using an additive printing device 1540. The lattice framework may then be integrated 1550 by an assembly machine with other elements of a foot cover such as an external shell or off the shelf midsoles or uppers. The customized foot cover with the lattice framework can then be shipped 1560 to the amputee.

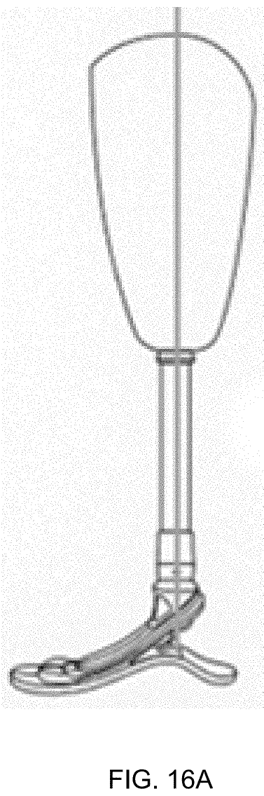

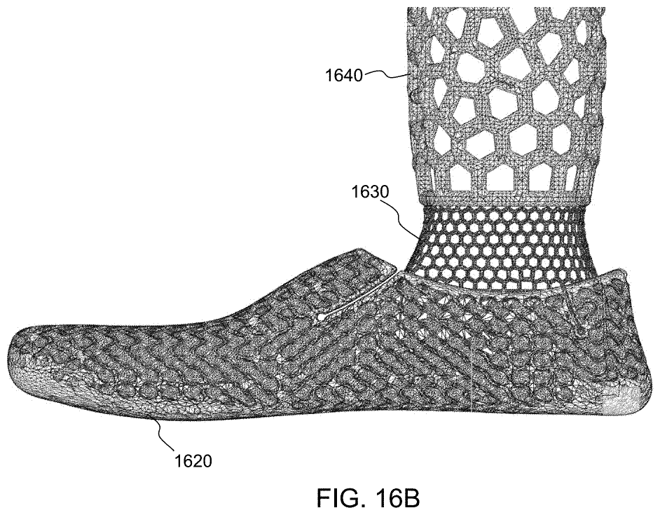

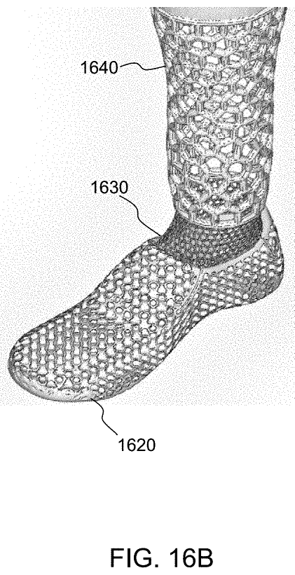

FIG. 16A illustrates an example of a prosthetic foot, ankle and/or leg that may be used with a lattice framework as a foot cover. Portions of the prosthetic foot, ankle and leg may use a lattice framework as a cover or a lattice framework may form parts of the prosthetic device. FIG. 16B illustrates a foot cover 1620 created from a lattice framework. In addition, a prosthetic ankle 1630 and/or foot is illustrated and can be made from a denser lattice framework. The prosthetic ankle 1630 or prosthetic foot may be contained within the foot cover 1620. Further, a portion of a prosthetic leg 1640 or even the remnant limb socket (not shown in this figure) may be made from a lattice framework. The prosthetic leg 1640 may have less dense lattice cells with thicker cell walls. FIG. 16C illustrates a perspective view of a foot cover 1620, prosthetic ankle 1630 and prosthetic leg 1640 using a lattice framework. Even though this technology described above refers to a foot, the same invention can be used in any type of prosthetic or robotic limb flexion device (e.g., any robotic joint). Alternatively, the lattice framework technology described may be used to form a prosthetic knee or the lattice framework may be used in a prosthetic device that connects to a prosthetic knee. The U.S. patent application Ser. No. 17/124,449, filed on Dec. 16, 2020, entitled, “PROSTHETIC FOOT COVER SYSTEM”, is herein incorporated by reference in its entirety for what the document teaches and describes.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A prosthetic foot cover device, comprising:
a lattice framework formed to replace a human foot;
an open cavity formed in the lattice framework which is sized and shaped to contain a prosthetic foot;
a pressure chamber, disposed in the lattice framework;
a first one-way check valve for the pressure chamber to allow air to be expelled from the pressure chamber and to generate a negative pressure within the pressure chamber as an amputee uses the prosthetic foot and the lattice framework; and
a remnant limb socket connected to the pressure chamber, wherein a negative pressure is developed in the remnant limb socket as air is pulled from the remnant limb socket into the pressure chamber.

2. The prosthetic foot cover device as in claim 1, further comprising a second one-way check valve for the pressure chamber to allow air to be drawn into the pressure chamber.

3. The prosthetic foot cover device as in claim 2, further comprising a first conduit to route air between the remnant limb socket and the second one-way check valve and a second conduit to route air between the first one-way check valve and outside atmosphere.

4. The prosthetic foot cover as in claim 1, wherein the lattice framework contains a sealed chamber in the lattice framework to provide cushioning for the prosthetic foot.

5. The prosthetic foot cover as in claim 4, wherein the sealed chamber is in proximity to at least one of: a plantar surface, a heel, a ball or a toe of the prosthetic foot to absorb shock from the prosthetic foot.

6. The prosthetic foot cover as in claim 5, wherein the sealed chamber is at least one of a sealed hollow chamber or a sealed pressurized chamber.

7. The prosthetic foot cover as in claim 1, further comprising an assistive negative pressure pump to withdraw air from the pressure chamber when a user applies load and compresses the pressure chamber.

8. The prosthetic foot cover as in claim 1, further comprising an outer membrane layer disposed over the lattice framework to cover and support the lattice framework.

9. The prosthetic foot cover as in claim 8, wherein the outer membrane layer is configured to be removable.

10. A prosthetic foot cover device, comprising:
a lattice framework formed to resemble a human foot;
an open cavity formed in the lattice framework which is sized and shaped to contain a prosthetic foot, wherein the open cavity has an opening to receive the prosthetic foot;
a pressure chamber, disposed in the lattice framework at a location where the prosthetic foot compresses the pressure chamber during an amputee's gait;
a first one-way check valve in fluid communication with the pressure chamber to allow air to be expelled from the pressure chamber to generate a negative pressure within the pressure chamber as an amputee uses the prosthetic foot and the lattice framework;
a second one-way check valve connected with the pressure chamber to allow air to be drawn into the pressure chamber; and
a remnant limb socket connected to the second one-way check valve, wherein a negative pressure is developed in the pressure chamber and remnant limb socket as air is pulled from the remnant limb socket through the second one-way check valve into the pressure chamber.

11. The prosthetic foot cover device as in claim 10, further comprising a conduit to route air between the remnant limb socket and the second one-way check valve.

12. The prosthetic foot cover as in claim 10, wherein the lattice framework contains a sealed pressurized chamber in the lattice framework to provide cushioning for the prosthetic foot.

13. The prosthetic foot cover as in claim 12, wherein the sealed pressurized chamber is in proximity to at least one of: a plantar surface, a heel, a ball or a toe of the prosthetic foot to absorb shock from the prosthetic foot.

14. The prosthetic foot cover as in claim 10, further comprising an assistive negative pressure pump to withdraw air from the pressure chamber when a user applies load and compresses the pressure chamber.

15. The prosthetic foot cover as in claim 10, further comprising a heel bumper chamber.

16. The prosthetic foot cover as in claim 15, further comprising air valves for the heel bumper chamber to enable pressure of the heel bumper chamber to be adjusted.

17. A method for additive manufacture of a prosthetic foot cover device, comprising:
receiving specifications at a computing device regarding use of the prosthetic foot including an amputee metric;
analyzing the amputee metric to define an architecture of lattice framework to be generated;
generating the lattice framework using an additive manufacturing printer; and
integrating the lattice framework into a prosthesis to form a customized prosthetic foot cover for the amputee.

18. The method as in claim 17, further comprising generating an outer membrane layer configured to be disposed over the lattice framework, wherein the outer membrane layer is printed using the additive manufacturing printer.

19. The method as in claim 17, wherein receiving specifications regarding use of the prosthetic foot further comprise receiving specifications from medical personnel.

20. The method as in claim 17, wherein the amputee metric further comprises an amputee metric selected from at least one of: physical user data obtained from a digital sensor, an amount of activity performed by the amputee, a weight of the amputee, a height of the amputee, or a measured gait of the amputee.

21. The method as in claim 17, further comprising, defining an architecture of lattice framework to be generated and used underneath at least one of: a sole of a foot, a ball of a foot or a heel of a foot based in part on the amputee metric.

22. The method as in claim 17, further comprising receiving specifications from at least one electronic sensor.
